(19) United States
(12) Patent Application Publication
(10) Pub. No.: US 2010/0069338 A1
(43) Pub. Date: Mar. 18, 2010

(54) PHARMACEUTICAL COMPOSITIONS

(75) Inventors: Simon J. Ward, Sheffield (GB); Alice MacGowan, Sheffield (GB); Michael J. Cork, Sheffield (GB); Adrian Davis, Sheffield (GB)

Correspondence Address: DAVIS WRIGHT TREMAINE LLP/Los Angeles, 865 FIGUEROA STREET, SUITE 2400, LOS ANGELES, CA, 90017-2566, US

(73) Assignee: YORK PHARMA PLC, Sheffield (GB)

(21) Appl. No.: 12/439,023

(22) PCT Filed: Aug. 31, 2007
(86) PCT No.: PCT/EP07/59102

§ 371 Date: Oct. 21, 2009

(30) Foreign Application Priority Data
Aug. 31, 2006 (GB) 0617191.2

(51) Int. Cl. A61K 31/19; A61P 17/00; A61K 31/194; A61K 31/191; A61K 31/195; A61K 31/60

(52) U.S. Cl. 514/159; 514/574; 514/557; 514/563

(57) ABSTRACT

The present invention provides a composition for topical administration to the skin for treating and/or preventing a dermatological condition involving an abnormal decrease in the cell-to-cell adhesion between epithelial cells comprising a zinc salt which is at least partially soluble in water and an acid, wherein the composition has a pH less than or equal to 6. The dermatological condition is generally selected from eczema, dermatitis, atopic eczema, atopic dermatitis, non-atopic eczema, non-atopic dermatitis, seborrheic eczema, irritant contact dermatitis, allergic contact dermatitis, pruritus and sensitive skin. A preferred composition comprises zinc lactate, lactic acid and has a pH of about 4.0 to 5.0.

### BACKGROUND

The present invention relates to the treatment, including prophylaxis, of dermatological conditions, compounds and pharmaceutical compositions for use in such treatments and the use of such compounds and compositions for the manufacture of medicaments for use in such treatments. The invention also relates to cosmetic compositions for application to the skin. In particular, the present invention relates to methods of repairing a disease or environmentally damaged skin or epidermal barrier, protecting the skin or epidermal barrier against damage or degradation by disease or environmental factors, compounds and compositions for use in such methods, and the use of such compounds and compositions for the manufacture of medicaments for use in the practice of such methods.

The present invention is particularly useful in the treatment of dermatological conditions involving an abnormal decrease in the cell-to-cell adhesion between epithelial cells, in particular between corneocytes. Examples of such dermatological conditions include various forms of eczema and dermatitis, such as atopic and non-atopic eczema or dermatitis, seborrheic eczema, irritant contact dermatitis, allergic contact dermatitis and other sensitive skin conditions, particularly those that cause or are associated with pruritus.

Such conditions, particularly atopic and non-atopic eczema or dermatitis, irritant contact dermatitis and sensitive skin, all arise partially or completely as a result of a defective skin or epidermal barrier.

Atopic eczema and irritant contact dermatitis are both characterised by a chronic phase when the skin is dry and slightly itchy with a defective epidermal barrier. The defective barrier permits the penetration of environmental triggers such as house dust mite faecal allergens and toxins released by the bacterium, Staphylococcus aureus. (Cork 1997 (3); Cork 1996 (2)). These allergens trigger a flare of atopic eczema that occurs because the allergens trigger the production of pro-inflammatory cytokines within the skin (Cork 1996 (2); Cork 1999 (5)).

Atopic eczema is a disease that usually starts before the age of two and causes enormous suffering at a crucial time in a child's development. Eczematous skin is characterised by dryness, erythema (redness), exudation and intense pruritus (itching). The itching associated with eczema leads to the child scratching their skin until it bleeds. The prevalence of atopic eczema has been increasing progressively over the past fifty years and now affects up to 30% of children (Williams 1992 (15), Thestrup-Pedersen 1996 (13), Cork et al., 2002 (6)). This rise has been accompanied by an increasing exposure to environmental agents such as soap, detergents and house dust mite (Cork et al., 2002 (6)), each acting to breakdown the skin barrier, an effect more pronounced in patients with atopic eczema (White et al., 1987 (14), Cork 1997 (3)).

The defective epidermal barrier in atopic eczema results in water loss from the corneocytes. The corneocytes shrink and cracks open between them, permitting the penetration of irritants and/or allergens and triggering the development of eczematous lesions.

Atopic eczema is predominantly a disease of childhood and 70% of children will “grow out” of their eczema by the time they are 16. However, in adult life they have a high probability of developing irritant contact dermatitis of the hands, the commonest occupational disease in Europe, as they remain genetically predisposed to barrier breakdown exacerbated by environmental stimuli (soap, detergent etc). The current management of irritant contact dermatitis of the hands consists of irritant avoidance, a complete emollient therapy regimen (Cork 1998 (4)) and topical steroids to treat flare-ups.

Sensitive skin is a condition that affects about 50% of the population, and manifests as burning, stinging and redness following the application of topical products such as cosmetics. Individuals who have sensitive skin may have had a history of atopic eczema.

Atopic eczema is a disease that has increased in prevalence from 4% of children in the 1940's to 30% of children at present. Atopic eczema is an example of multifactorial disease that arises as a result of the interaction of changes in several genes with multiple environmental factors. The genetic basis of atopic eczema has not changed over the past 60 years, but there have been several changes in our environment, these include increased washing with soap and detergents and increased exposure to house dust mites (Cork et al.: 2003 (7)).

### Existing Treatments for Atopic Dermatitis or Eczema, Irritant Contact Dermatitis and Like Skin Conditions

### (i) Complete Emollient Therapy (Best Practice)

A regimen consisting of emollient cream/ointments, emollient soap substitutes and bath and shower emollients. These products replace all soap and detergents and as a result produce a reduction of environmental damage to the skin barrier (Cork 1997 (3), Cork 1998 (4), Cork 1999 (5)). Examples of leading emollient products in Europe include: Cream E45, Diprobase®, Hydromol®, Lipobase® and Oilatum®.

Emollients produce a partial repair of the skin barrier but because of the defective corneodesmosomes, associated with these diseases, irritants and allergens can still penetrate through the skin and trigger a flare of the eczema. Moreover, because it involves frequent applications, patients find emollient therapy highly inconvenient and compliance with treatment regimens, therefore, can be poor.

### (ii) Topical Corticosteroids

These drugs are used to treat a “flare-up” of atopic eczema. The principle adverse effects of topical steroids are cutaneous atrophy and adrenal axis suppression. These adverse effects are predominantly associated with potent and very potent topical corticosteroids. Steroid paranoia, or steroid phobia, is a significant cause of poor or non-compliance with treatment regimens and is a major problem with all corticosteroids (Charman et al., 2000 (1)).

(iii) Calcineurin Inhibitors, Tacrolimus (Protopic®: Fujisawa) and Pimecrolimus (Elidel®: Novartis)

These drugs represent a new class of treatment for flare-ups of atopic eczema. They produce a selective inhibition of T cells via the calcineurin pathway. The calcineurin inhibitors do not cause cutaneous atrophy, nor do they have any effect on the adrenal axis. However, whilst treatment with calcineurin inhibitors can control flares of atopic eczema, they do not correct the underlying defects in the epidermal barrier, which allow it to be penetrated by the irritants and allergens that cause such flares.

There is a need, therefore, for new topical products that can repair or prevent damage to the epidermal barrier, for their use would reduce the degree to which it can be penetrated by irritants and allergens, and the frequency or severity of any consequential flares of atopic eczema that require drug treatment.

An object of the present invention is to provide a treatment for dermatological conditions involving an abnormal decrease in the cell-to-cell adhesion between epithelial cells (in particular between corneocytes), especially for eczema, dermatitis and like conditions, such as atopic and non-atopic eczema or dermatitis, seborrheic eczema, irritant contact dermatitis, allergic contact dermatitis and other sensitive skin conditions, particularly those that cause or are associated with pruritus, that does not suffer from the drawbacks of the aforementioned known treatments. In particular, it is an object of the invention to provide a treatment that is convenient, does not involve a complex treatment regimen, or the use of drugs with the potential to cause side effects of the nature associated with the prolonged use of topical corticosteroids.

### SUMMARY OF THE INVENTION

A first aspect of the present invention provides a composition for topical administration to the skin for treating and/or preventing a dermatological condition involving an abnormal decrease in cell-to-cell adhesion between epithelial cells comprising a zinc salt which is at least partially soluble in water and an acid, wherein the composition has a pH less than or equal to about 6.

A second aspect of the present invention is a method for treating and/or preventing a dermatological condition involving an abnormal decrease in cell-to-cell adhesion between epithelial cells comprising topical administration of a composition comprising a zinc salt which is at least partially soluble in water and an acid, wherein the composition has a pH less than or equal to 6.

A third aspect of the present invention provides a composition comprising zinc lactate, lactic acid and wherein the composition has a pH of about 4.0 to 5.0.

A fourth aspect of the present invention provides materials, comprising zinc oxide and an acid, from which a composition for topical administration to the skin for treating and/or preventing a dermatological condition involving an abnormal decrease in cell-to-cell adhesion between epithelial cells comprising a zinc salt which is at least partially soluble in water and an acid, wherein the composition has a pH less than or equal to about 6 can be generated.

### BRIEF DESCRIPTION OF THE FIGURES

FIG. 1 shows the epidermal barrier in which corneodesmosomes [1], covalently bound lipid [2], extension of lamellar granule lipids [3], lipid bilayers [4], keratohyalin granules [5] and desmosomes [6] are shown.

FIG. 2 on the left hand side provides a another schematic representation of the skin in which the layers stratum corneum [21], stratum granulosum [22], stratum spinosum [23] and stratum basale [24] are shown. The location of desmosomes [31] and corneodesmosomes [30] are also shown. On the right hand side FIG. 2 shows the components of a corneodesmosome including intermediate filaments [25], desmoglein [26] and desmocollins [27], plakoglobin [28] and desmoplakins [29]. FIG. 2 provides a schematic representation of the skin and shows the barrier to the penetration of irritants, allergens and drugs is located in the lower part of the stratum corneum. The structural integrity of the stratum corneum is maintained by modified desmosomes (corneodesmosomes) which lock together the corneocytes. As the corneocytes move up through the stratum corneum, the corneodesmosomes are gradually broken down by the skin-specific proteases and unattached corneocytes can then be desquamated from the surface of the skin.

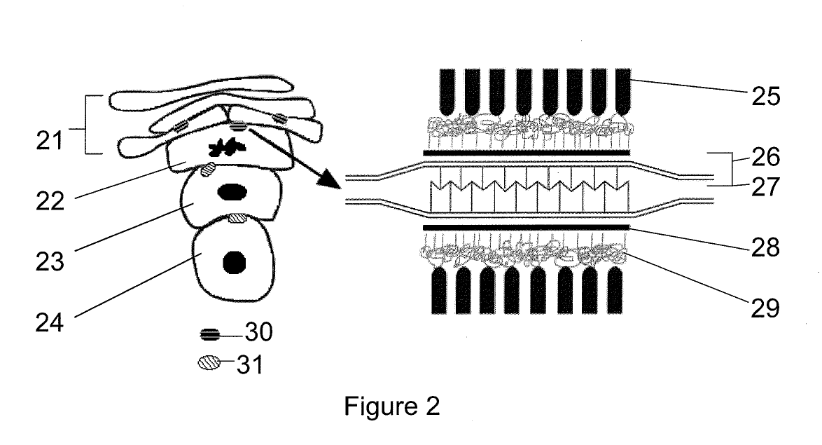

FIG. 3 shows the results of a study to compare the effects of a composition of the present invention against the effects of no treatment.

FIG. 4 shows the results of a study to compare the effects of a composition of the present invention against the effects of another proprietary emollient.

### DETAILED DESCRIPTION

The majority of research into the causes of atopic eczema over the past 50 years has focused on the development of IgE mediated allergic responses. However, the majority of children with atopic eczema are not immunologically atopic (Murphy et al., 1999 (12), Flohr et al., 2004 (9)). We have focused, therefore, on the skin or epidermal barrier as a primary site for the development of atopic eczema.

The barrier to the penetration of irritants and allergens into the skin is located in the stratum corneum. At the same time this barrier prevents the loss of water from the host and thereby maintains the internal homeostasis (Cork 1997 (3)).

The stratum corneum can be visualised as being rather like a brick wall with the corneocytes forming the bricks and the lamellar lipids the mortar (Elias 1983 (8)).

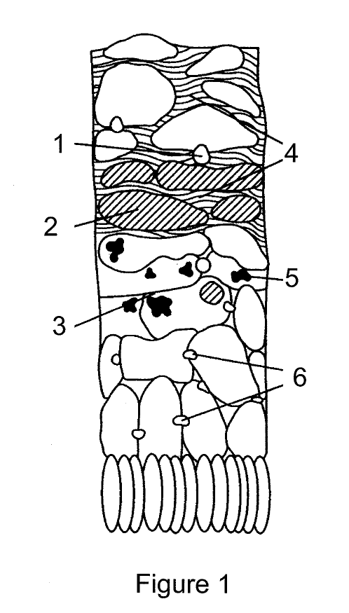

The corneodesmosomes lock the corneocytes together and prevent shearing forces dislodging the corneocytes (see FIG. 1). The corneodesmosomes can be visualised as analogous to the iron rods, which are passed down through holes in bricks to lock them together and add tensile strength to a brick wall.

Corneocytes are shed from the surface of the skin by a process of proteolysis, which is mediated by skin specific proteases, such as the stratum corneum chymotryptic enzyme (SCCE). These proteases are inhibited by skin specific protease inhibitors, such as the secretory leucocyte protease inhibitor (SLPI). It is essential for the process of desquamation to be tightly regulated in order to prevent premature desquamation and a breakdown of the skin barrier. A breakdown/thinning of the stratum corneum will permit the penetration of irritants and allergens, which in turn can lead to the development of flares of atopic eczema.

The integrity of the stratum corneum epidermal barrier is maintained by a balance between the levels of skin proteases, such as SCCE, the protease inhibitors, such as SLPI, and the vulnerability of the adhesion proteins, such as corneodesmosin, to the action of the proteases. The situation is complex as there are more than 10 adhesion proteins, 8 proteases and 10 protease inhibitors.

In normal skin, the breakdown of cellular adhesion proteins (e.g. corneodesmosin) during desquamation is regulated by a balanced expression of proteases (e.g. SCCE) and protease inhibitors (e.g. SLPI).

The skin has an acidic pH (the acid mantle) that contributes the optional barrier function of this tissue, which in healthy skin is around 5.4 to 5.9. The acid mantle has several effects; firstly, a strong antimicrobial effect and secondly, skin surface pH has a role on desquamation, permeability barrier homeostasis and stratum corneum integrity/cohesion.

In individuals who are genetically predisposed to atopic dermatitis, there is an increased expression of proteases, such as SCCE, and/or a decreased expression of protease inhibitors, such as SLPI, which leads to a premature breakdown and thinning of the epidermal barrier, allowing the penetration of irritants and allergens.

Endogenous proteases such as SCCE have a role to play in conditions involving an abnormal decrease in cell-to-cell adhesion between epithelial cells in the epidermal barrier.

Two of the environmental agents associated with atopic eczema, house dust mites and staphylococcus aureus, produce proteases, which can break the skin barrier down from the outside. Additionally, proteases produced as a product of the inflammatory response to irritants and allergens (eg mast cell chymase which is a secondary protease) are also capable of damaging the skin barrier. It therefore appears that the skin barrier is being broken down by endogenous, exogenous and secondary proteases in atopic eczema.

The most common environmental agents that increase the pH of the skin's surface are soaps and other detergents. It has been found that washing with soap can cause thinning of the stratum corneum in both healthy skin and the skin of individuals with dermatological conditions associated with reduced cell-to-cell adhesion between epithelial cells. Detergents are widely used in cleaning human skin. They work by emulsifying the skin's surface lipids (both foreign and natural), which can then be washed off with water.

Atopic eczema, therefore, is a classic example of a gene-environment interaction disease. Multiple environmental factors interact with changes in many genes to produce the disease phenotype.

The processes involved in irritant contact dermatitis can be essentially the same as those involved in atopic eczema. However, in some cases of irritant contact dermatitis, the abnormal decrease in the cell-to-cell adhesion between the epithelial cells in the epidermal barrier, which underlies the condition, can be caused entirely by an environmental insult, such as exposure to an environmental irritant. In such cases, sufferers need not have had a genetic predisposition towards developing the condition.

The processes involved in all of the conditions to which the present invention relates all involve breakdown of the skin or epidermal barrier through similar mechanisms to those involved in both atopic eczema and irritant contact dermatitis. They are multi-factorial diseases.

The present invention is based on an understanding of the various factors which interact and lead to dermatological conditions involving an abnormal decrease in the cell-to-cell adhesion between epithelial cells and has led to the development of topically applied compositions capable of treating and/or preventing (including the prophylaxis of) these conditions by causing a plurality of changes in the skin barrier.

The present invention provides a general purpose protease inhibitor that can antagonise many of the proteases which have a role in conditions such as atopic dermatitis, which involve an abnormal decrease in the cell-to-cell adhesion between epithelial cells. This has been achieved by the development, for the first time, of a formulation which can deliver zinc ions to the stratum corneum in the context of an acidic composition. This combination approach has not been achieved previously. Consequently, the present invention is a composition that both delivers zinc ions to the stratum corneum and lowers the pH of the stratum corneum. The compositions comprise an at least partially soluble zinc salt and a dermatologically absorbable acid.

The composition of the invention increases the solubility of the zinc ions and facilitates their penetration into the stratum corneum. The acid of the composition enhances both the solubility and the penetration of zinc.

Zinc ion delivery, ie penetration into the stratum corneum, is important for several reasons.

Firstly, there is plenty of evidence to demonstrate that zinc ions inhibit certain enzyme reactions and have potent anti-protease activity. SCCE, which is of particular interest due to its role in corneocyte desquamation, is a serine protease. It is established that this class of proteases is inhibited by zinc ions. Zinc may also stabilize the binding of protease inhibitors to the active site, thereby increasing their efficacy.

Secondly, zinc ions have an anti-microbial activity. As described above Staphyloccous aureus is involved in both chronic atopic dermatitis as well as flare-ups of the condition. The anti-microbial effect of zinc ions can treat and/or prevent conditions of this type, including by preventing release of exogenous proteases from microbes that damage the skin barrier.

Thirdly, a zinc ion deficiency, caused by impaired absorption of zinc from the gastrointestinal tract leads to the condition acrodermatitis enteropathica. This condition is characterised by eczematous skin lesions, alopecia and diarrhoea, and can also be a precursor of other conditions involving an abnormal decrease in cell-to-cell adhesion between epithelial cells. Topical administration of zinc has been shown to cause a rapid improvement in symptoms of acrodermatitis enteropathica.

Lowering the pH of the stratum corneum is important for the following reasons. It has been found that the skin protease SCCE, which is involved in corneocyte desquamation, has a neutral pH optimum and a change in pH from 7.5 to 5.5 reduces SCCE activity by 50%. Therefore lowering the pH of the stratum corneum moves the pH away from the optimum pH of skin protease enzymes such as SCCE and therefore their activity decreases.

The lipid-generating enzymes β-glucocerebrosidase and sphingomyelinease which are involved in formation of the lipid lamellae, have acid pH optima. Therefore lowering the pH within the stratum corneum moves the pH towards the optimum for these enzymes.

Lowering the pH within the stratum corneum also enhances the antimicrobial effect of the skin.

Furthermore, a low pH has an anti-bacterial effect. This can help prevent damage to the stratum corneum by bacteria such as staphylococcus aureus, which is known to be involved in conditions involving an abnormal decrease in cell-to-cell adhesion of epithelial cells.

For the compositions of the present invention it is necessary to select a zinc salt with an appropriate solubility level. The zinc salt chosen must be at least partially soluble in water in order for delivery to the stratum corneum to be achieved. However, a very highly soluble zinc salt may lead to local toxicity, unless used at a carefully selected low concentration.

The penetration of zinc ions can be measured using atomic absorption spectroscopy.

A zinc salt which is at least partially soluble in water has a solubility in water of at least 0.01 moles/l, preferably at least 0.05 moles/1, more preferably at least 0.1 moles/1. A preferred solubility for a zinc salt is about 0.2 moles/l. In embodiments an upper solubility level of the zinc salt can be 0.4 moles/1 or 0.6 moles/l. In embodiments the zinc salt has a solubility in the range of 0.01 moles/1 to 0.6 moles/l, preferably in the range of 0.05 moles/1 to 0.4 moles/l. As the skilled person would be aware, the solubility is measured at room temperature.

The zinc salt chosen may be a salt of an alpha-hydroxy carboxylic acid or a carboxylic acid.

Suitable zinc salts for use in the compositions of the present invention include: zinc citrate, zinc glycerate, zinc glycolate, zinc picolinate, zinc tartrate, zinc pantothenate, zinc lactate, zinc gluconate, zinc pyruvate, zinc salicylate, zinc formate, zinc formate, zinc acetate, zinc propionate, and zinc butyrate.

Preferred zinc salts are zinc lactate, zinc citrate and zinc glycolate.

A particularly preferred zinc salt is zinc lactate because zinc lactate has a humectant activity. Zinc lactate mimics compounds present in natural moisturising factor. Furthermore zinc lactate has antimicrobial effects and this activity is beneficial because it reduces the damage to the stratum by another route.

The percentage of zinc salt in compositions of the present invention is at least about 0.5%, preferably at least about 1%. Preferred zinc salt concentrations are about 1%, preferably about 1.5% and more preferably about 2%, or about 2.5% or about 3%. In embodiments an upper limit on the percentage of the zinc salt present in the composition is 20%, 15%, 10%, 7.5% or 5%.

The acid chosen for compositions of the present invention is one which is dermatologically absorbable, meaning one which penetrates into the stratum corneum sufficiently to have the desired effect. The acid chosen can be absorbed into the stratum corneum. Once absorbed into the stratum corneum it can lower the pH there.

The penetration of acids into the stratum corneum can be measured by taking serial skin strippings with adhesive tape and measuring the acid content of the corneocytes which are retained on the strips using HPLC. Measurement of the acid levels within the skin may also be measured using other biophysical measurements on the skin including infrared spectroscopy.

An acid which is not absorbable into the skin would not be chosen. An example of this would be fatty acids including linoleic acid.

Due to the presence of an acid, the compositions of the present invention have an acidic pH. The pH of the composition should be equal to or less than about 6. Thus the pH of the composition can be about equal to or less than about 6.0, preferably equal to or less than about 5.5, optionally equal to or less than about 5.0. The range of pH for the compositions of the invention is about 3 to about 5.5. The preferred range of pH for the compositions of the invention is about 4.0 to about 5.5. Most preferably the compositions of this invention have a pH of about 4.0 or about 4.5 or about 5.0 or about 5.5.

As noted above the purpose of the present invention is to enable delivery of zinc ions to the stratum corneum in the context of an acidic pH. One of the reasons for requiring an acidic pH is because a high pH could neutralize the action of the zinc ions. The low pH of the compositions of the present invention enhances the penetration of zinc ions into the skin and also enhances the inhibitory activity of the zinc ions.

The acid chosen may be an organic acid, an alpha-hydroxy carboxylic acid or a carboxylic acid.

Preferred acids for use in compositions of the present invention include: citric acid, glyceric acid, glycolic acid, picolinic acid, tartaric acid, pantothenic acid, lactic acid, gluconic acid, pyruvic acid, salicyclic acid, formic acid, acetic acid, propionic acid and butyric acid.

A particularly preferred acid is lactic acid because it is naturally involved in the skin's pH gradient. Lactic acid is also well absorbed into the skin, which is key to it reaching the stratum corneum to deliver its effect. Lactic acid is also part of natural moisturising factor and it is deficient in people with eczema.

The anion of the zinc salt used in compositions of the present invention may be the same or different from the acid residue used in the compositions.

A preferred embodiment of the present invention is a composition comprising zinc lactate as the zinc salt, lactic acid as the dermatologically absorbable acid and has a pH in the range 4.0 to 5.5, and preferably pH 4.0.

The composition preferably also includes a pharmaceutically acceptable carrier or vehicle and is for topical application to the skin.

In embodiments the zinc salt in the compositions of the present invention may be generated during the formulation of the compositions. The zinc salt can be generated from zinc oxide and an acid. In the present invention a preferred zinc salt is zinc lactate, which can be generated from zinc oxide and lactic acid.

The dermatological conditions to be treated in accordance with the various aspects of the invention are preferably those involving an abnormal decrease in the cell-to-cell adhesion between epithelial cells, in particular between corneocytes, in the epidermal (or skin) barrier, particularly in the stratum corneum. The abnormal decrease in the cell-to-cell adhesion between the epithelial cells in the epidermal barrier, which underlies these conditions, can be caused by one or more of many factors. These include genetically determined factors, such as those described above with reference to atopic eczema, and environmental insults, such as exposure to an environmental irritant. Examples of dermatological conditions that can be treated in accordance with the present invention include eczema, dermatitis and like conditions, such as atopic and non-atopic eczema or dermatitis, intrinsic atopic eczema or dermatitis, extrinsic atopic eczema or dermatitis, seborrheic eczema, irritant contact dermatitis, cosmetic eczema or dermatitis, allergic contact dermatitis, dry skin, dry/sensitive skin and other sensitive skin conditions, particularly those that cause or are associated with pruritus. Preferred examples of dermatological conditions that can be treated in accordance with the present invention include irritant/allergic contact eczema or dermatitis of the hands and allergic/irritant contact facial eczema or dermatitis. The prevention or prophylaxis of dermatological conditions resulting from exposure to an excess of ultraviolet light, including sunburn, are excluded from the ambit of certain preferred embodiments of the present invention, as is the use of any composition in accordance with the invention as a sun screen or for the treatment of diaper rash.

It has recently been suggested that the cutaneous manifestations of atopy, particularly atopic eczema or dermatitis (in the literature, the terms atopic dermatitis and atopic eczema are used interchangeably) often represent the beginning of the atopic march (Spergel and Paller 2003 (16)). It has been shown in several longitudinal studies that approximately half of the infants who suffer from atopic eczema or dermatitis will develop asthma, particularly those with severe atopic eczema, and two-thirds will go on to develop allergic rhinitis (hay fever). Epicutaneous sensitisation is thought to be responsible, with subsequent migration of sensitised T cells into the nose and airways, causing these upper and lower airway diseases. Animal models and human observations concur with this theory and preliminary prevention studies with oral antihistamines have provided evidence that early intervention might slow the atopic march (Spergel and Paller 2003 (16)).

During the first six months of a baby's life, the immune system is ‘malleable’ and can be more easily switched from TH1 to TH2. It is therefore possible that the defective skin barrier in a baby with atopic eczema or dermatitis is allowing the penetration of allergens at a crucial time, causing TH1 to TH2 switching, and that this immunological change results in a predisposition to more severe allergic eczema, asthma and hay fever. If the skin barrier were to be restored during the first six months of life, therefore, this TH1 to TH2 switching could be prevented or reduced and with it the atopic march and the development of atopic eczema, asthma and hay fever.

Accordingly, in a further aspect of the present invention, compositions in accordance with the invention can be employed in methods of slowing or preventing the atopic march, reducing TH1 to TH2 switching and/or controlling the development, preventing, reducing the risk of development, and/or prophylaxis of upper and lower airways diseases, particularly asthma and allergic rhinitis. In the practice of this method, compositions in accordance with the invention are used to treat atopic eczema or dermatitis, particularly in young children and infants under the age of about 5, 3, 2, 1 years or, preferably, six months. Methods of treating airway diseases in this manner also fall within the ambit of the present invention.

In a yet further aspect of the invention, compositions in accordance with the invention can be used as protease antagonists and to thereby prevent or control pruritus. Methods of controlling pruritus in this manner are also within the ambit of the present invention.

In preferred embodiments, compositions in accordance with the invention are formulated for topical application to the skin. In this regard, the inventive compositions can include one or more pharmaceutically acceptable excipient, carrier, diluent or vehicle and can be in the form of a soak, ointment, cream, lotion, paste, gel, stick, spray, aerosol, bath oil, shampoo, soap, foam, spray or solution.

Preferred excipients include those that provide emolliency, skin hydration via humectancy and occlusion and aesthetic qualities to improve compliance, these are consistent with the overall therapeutic objectives of the invention. Thus a range of oils and lipids, including petrolatum, light liquid paraffin, mineral oils, fatty acid derivatives including isopropyl myristate, isopropyl palmitate and such, glycerides, natural oils, lanolins such as Medilan, are suitable. A preferred oil phase is petrolatum-mineral oil as this gives maximum occlusion percent of composition.

A range of glycol or other humectants suitable to hydrate the skin include, glycerol, propylene glycol, polyethylene glycol (100-1000), sodium pyrrolidone carboxylic acid and the like. A preferred humectant is glycerol as this gives maximum humectancy percent of composition.

Volatile and non-volatile silicone, alkyl esters and other such material known in the art may be added to improve skin feel and patient compliance.

Surfactants, waxes and gelling agents may be added to provide suitable rheology. Preservative may be added to provide microbiological quality.

When used in this specification to qualify the nature of a compound or composition, the term “pharmaceutically acceptable” means that the compound, or composition, in question does not cause significant side effects in a significant number of patients, when it is used in a quantity, or concentration at which it has the promised therapeutic effect. All references in this specification to the treatment of diseases, conditions and the like encompass prophylaxis or preventative treatments, as well as therapeutic or curative use.

The following examples are provided by way of illustration only and are not intended to in any way limit the scope of the present invention.

### EXAMPLES

### Example 1

The following table sets out a preferred composition in accordance with the present invention.

| Ingredient | | % in composition |
|---|---|---|
| Function | Ingredient name | of Example 1 |
| Active | Zinc Lactate | 2 |
| | (Lactic acid to pH 4.00) | |
| Solvent/Humectant | Water | 34 ~(to 100) |
| | Glycerine | 15.0 |
| Emollient/Barrier | Pet-mineral oil | 30.0 |
| | Dimethicone | 3.0 |
| | Cyclomethicone | 2.0 |
| Structuring | Glyceryl Stearate | 2.0 |
| | Methylglucose sesquistearate | 4.0 |
| | PEG20- Methylglucose | 2.0 |
| | sesquistearate | |
| | Cetyl Alcohol | 1.5 |
| | Stearic acid | 1.5 |
| | Xanthan Gum | 0.5 |
| | Magnesium Al Silicate | 0.25 |
| | Sepigel | 0.25 |
| Preservation | Benzyl alcohol | 2.0 |

The composition is made by heating oil (emollient and structuring components) and water (actives and solvent/humectant) phase components separately to 75° C. Mixing whilst stirring intensively. Cooling to room temperature whilst stirring, and adding preservative mixture.

This composition was tested to assess its effectiveness. Two volunteers took part in a hand washing dry skin model to assess the effect of the composition of Example 1.

The hand washing dry skin model was selected because it produces eczematous lesions that are similar to those found in atopic eczema. Washing the skin with soap raises the pH of the skin from the normal of 5.5 to at least 7.5. The skin proteases such as SCCE have a pH optima of 7.5 and so by washing with soap this enhances the protease activity in the skin in a similar manner to that seen in atopic eczema. This is a model system because the individuals used for the study do not actually have atopic eczema. This model provides an ideal system to make functional assessments of the skin following the application of a topical skin protease inhibitor cream.

In each study a volunteer washed their hands ten times per day with a soap for one week. This process induces dry skin and in some individuals it may also induce erythema (redness) and cracking. These clinical changes in the appearance of the skin are recorded after the completion of the one week of hand washing with soap and then again after the treatment period with the composition of Example 1, or the control.

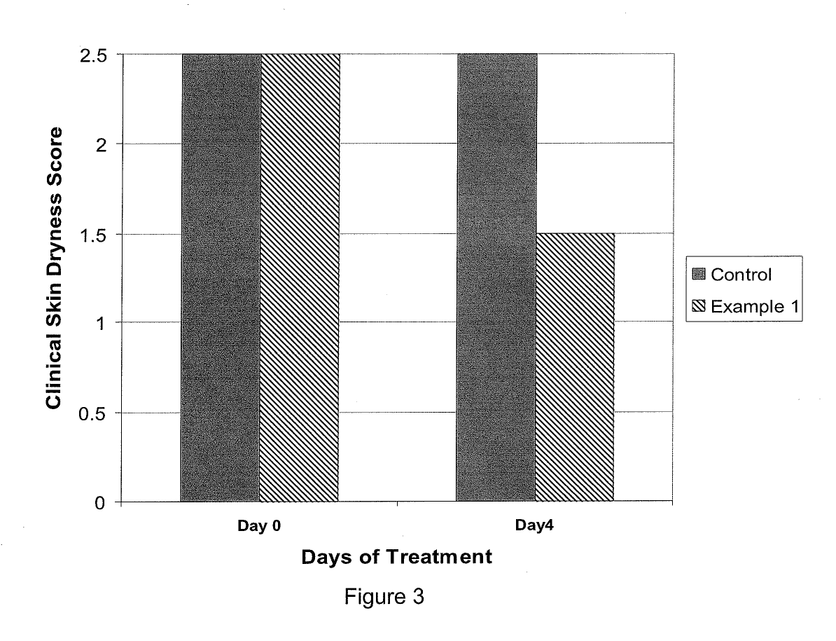

In the first study one hand of the volunteer was treated six times a day, for four days with the composition of Example 1. The other hand received no treatment. The results are shown in FIG. 3. In which the y-axis shows the clinical skin dryness score. This score records the skin dryness on the hands of the volunteers as assessed by a dermatologist. The following scale is used:
0=>no dryness
1=>slight dryness
2=>moderate dryness
3=>severe dryness
4=>very severe dryness

At base line, the skin dryness scores were 2.5 for both the hands treated with the composition of Example 1 and the untreated hands (FIG. 3). After four days treatment with the composition of Example 1 the skin dryness score had fallen from 2.5 to 1.5. The untreated, control, hand skin dryness was unchanged at 2.5, after the same four days.

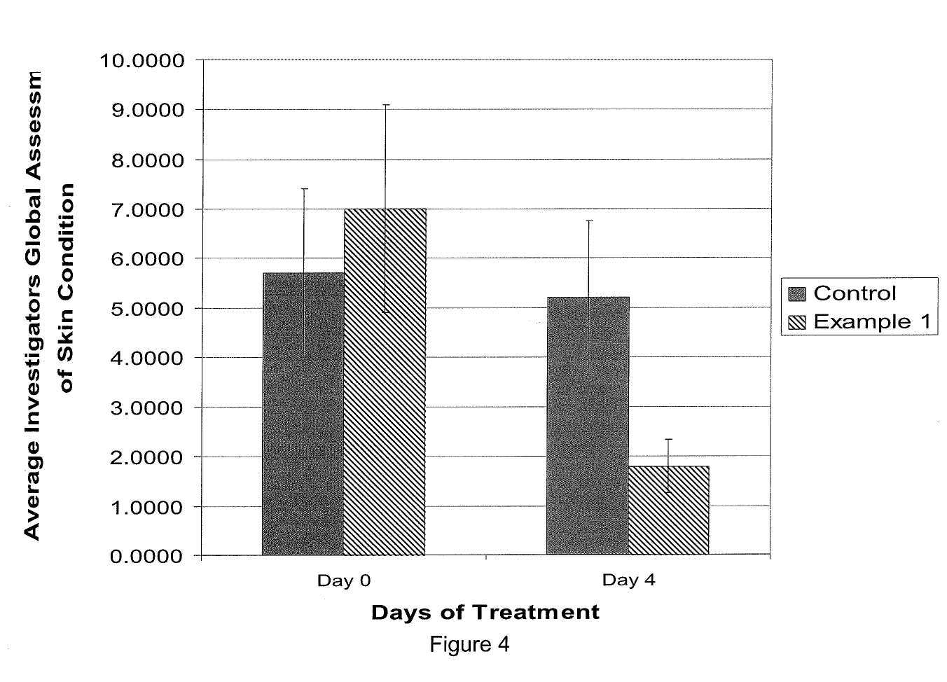

In the second study, one hand of the volunteer was treated six times a day for four days with the composition of Example 1. The other hand was treated with a comparator emollient (Diprobase®). The results are shown in FIG. 4, in which the y-axis shows the investigators global assessment (IGA) of the clinical condition of skin as assessed by a dermatologist. The investigators global assessment score is a combination of skin dryness and erythema measurements using the following scales:
Skin dryness: 0=>no dryness
1=>slight dryness
2=>moderate dryness
3=>severe dryness
4=>very severe dryness
Skin erythema (redness): 0=>no erythema
1=>slight erythema
2=>moderate erythema
3=>severe erythema
4=>very severe erythema

The results in FIG. 4 show that at baseline, day 0, the average (of 3 sites) investigators global assessment on the control skin site was 5.7 and the site to be treated with the composition of Example 1 was 7.0. After four days treatment with composition of Example 1 the IGA had fallen to 1.8 (from 7.0). The control had fallen from 5.7 to 5.2 during the same period.

The cosmetic acceptability of the composition of Example 1 has also been tested. The composition of Example 1 was designed to be acceptable to patients with moderate severity atopic eczema. Patients in this group will prefer an emollient that is in between the lightest proprietary products such as hydromol cream and the heaviest emollient products such as 50% light liquid paraffin/50% white soft paraffin. Evaluations of the cosmetic acceptability of the composition show positive responses from the volunteers tested.

### Example 2

### A Cream Example

### Emulsion with Glucam E20

| Ingredient | | % in composition |
|---|---|---|
| Function | Ingredient name | of Example 2 |
| Active | Zn lactate | 1.0 |
| | Lactic acid | (to pH 4.0) |
| Solvent/humectant | Water | (to 100) |
| | Glycerin | 5.00 |
| Emollient/Barrier | Pet-mineral oil | 9.0 |
| | Isohexadecane | 10.00 |
| Structuring | Glyceryl Stearate, | 6.00 |
| | PEG-100 Stearate | |
| | Stearic Acid | 2.00 |
| | PPG-15 Stearyl | 3.00 |
| | Ether | |
| | Sorbeth-30 | 4.00 |
| | Methyl Gluceth-20 | 4.00 |
| Preservation | Kemaben 4: | 0.60 |
| | Phenoxyethanol, | |
| | Methylparaben, | |
| | Butylparaben, | |
| | Ethylparaben, | |
| | Propylparaben | |

This composition was made as follows. Heat oil (emollient and structuring components) and water (actives and solvent/humectant) phase components separately to 75° C.

Mix whilst stirring intensively.

Homogenise for 1 minute at 75° C.

Cool to room temperature whilst stirring and add preservative mixture

### Example 3

### A Further Cream Example

### Emulsion with Glucam-Glucate-Glucamate

| Ingredient | | % in composition |
|---|---|---|
| Function | Ingredient name | of Example 3 |
| Active | Zn lactate | 2.0 |
| | Lactic acid | (to pH 4.5) |
| Solvent/humectant | Water | (to 100) |
| | Glycerin | 15.00 |
| Emollient/Barrier | Pet-mineral oil | 30.00 |
| | Isohexadecane | 5.00 |
| Structuring | Methyl Glucose | 1.50 |
| | Sesquistearate | |
| | PEG-120 Methyl | 2.00 |
| | Glucose Sesquistearate | |
| | Methyl Gluceth-20 | 4.00 |
| | Stearic Acid | 2.00 |
| | Sorbeth-30 | 4.00 |
| Preservation | Kemaben 4: | 0.60 |
| | Phenoxyethanol, | |
| | Methylparaben, | |
| | Butylparaben, | |
| | Ethylparaben, | |
| | Propylparaben | |

This composition was made as follows. Heat oil (emollient and structuring components) and water (actives and solvent/humectant) phase components separately to 75° C.

Mix whilst stirring intensively.

Homogenise for 1 minute at 75° C.

Cool to room temperature whilst stirring and add preservative mixture.

### Example 4

A further preferred example of a composition in accordance with the present invention has the following ingredients and formulation process.

| Material | % w/w | INCI |
|---|---|---|
| Water | 37.69 (to 100.0) | aqua |
| Petroleum Jelly | 30.00 | petrolatum |
| Glycerin BP | 15.00 | glycerin |
| Silicone DC Q7-9120 | 4.00 | dimethicone |
| (DC200/350cs EP grade | | |
| Cetyl Alcohol | 2.00 | cetyl alcohol |
| Glucamate SSE 20 | 2.00 | PEG-20 Methyl Glucose |
| | | sesquistearate |
| Lasemul 92N40 | 2.00 | glyceryl stearate |
| Glucate SS | 1.50 | Methyl glucose |
| | | sesquistearate |
| Optiphen | 1.50 | phenoxyethanol/capylyl |
| | | glycol |
| Zinc Oxide | 1.00 | equivalent to Zinc lactate |
| *Purac FCC80 | 2.76 (2.21/0.8) | at 2.99%, see footnote |
| Keltrol TF(E) | 0.30 | xanthan gum |
| *Purac FCC80 | ~0.25 to pH 4.50 | excess lactic acid to pH |
| | | 4.50 |
| Total | 100.00 | |

*this same ingredient listed twice to distinguish the functions to:
form Zinc lactate in situ (1% ZnO + 2.76 Purac FCC80)
buffer to pH 4.5 (*0.25% Purac FCC80)

The composition was formulated as follows.

### Premix 1:

add water to weight to suitable vessel
add glycerine to weight to vessel
add zinc oxide to weight
add lactic acid to Zn lactate equivalent (ratio 1:2.76)
add excess lactic acid (0.25%)
   stir for 2 hours to dissolve all the zinc oxide
add xanthan gum

### Premix 2:

add petroleum jelly to weight to suitable vessel
add silicone DC to weight to vessel
add cetyl alcohol to weight to vessel
add glutamate SSE 20 to weight to vessel
add Lasemul to weight to vessel
add Glucate SS to weight to vessel
add Optiphen to weight to vessel

### Main Process

heat premix 2 to 60 c and stir to dissolved waxes
add heated premix 1 (60 c) to heated-dissolved premix 2 with appropriate emulsification
add lactic acid to pH 4.50
cool and pack
   Formation of Zinc Lactate from Zinc Oxide and Lactic Acid

ZnO+2H(lactate)>Zn(lactate)2+H2O

81.37+180.16=243.51+18

1+2.21=2.99+0.22

### Check: 3.21=3.21

The resulting composition has following final formulation

| Material | % w/w | INCI |
|---|---|---|
| Water | 37.69 (to 100.0) | aqua |
| Petroleum Jelly | 30.00 | petrolatum |
| Glycerin BP | 15.00 | glycerin |
| Silicone DC Q7-9120 | 4.00 | dimethicone |
| (DC200/350cs EP grade | | |
| Cetyl Alcohol | 2.00 | cetyl alcohol |
| Glucamate SSE 20 | 2.00 | PEG-20 Methyl Glucose |
| | | sesquistearate |
| Lasemul 92N40 | 2.00 | glyceryl stearate |
| Glucate SS | 1.50 | Methyl glucose |
| | | sesquistearate |
| Optiphen | 1.50 | phenoxyethanol/ |
| | | capylyl glycol |
| Zinc Lactate | 2.99 | Zinc Lactate |
| Keltrol TF(E) | 0.30 | xanthan gum |
| Purac FCC80 | ~0.25 to pH 4.50 | excess lactic acid to |
| | | pH 4.50 |
| Total | 100.00 | |

### REFERENCES

(1) Charman C R, Morris A D, Williams H C. Topical corticosteroid phobia in patients with atopic eczema. Br J Dermatol 2000; 142: 931-6
(2) Cork M. J. (1996) The role of staphylococcus aureus in atopic eczema: treatment strategies. JEADV 7(7): S31-S37.
(3) Cork M J. The importance of skin barrier function. J Dermatol Treat 1997; 8: S7-S13
(4) Cork M J. Complete Emollient Therapy. The National Association of Fundholding Practices Year book 1998, pp 159-68.
(5) Cork M J, Butler L, Young S et al. An audit of the effect of explanation and demonstration of topical therapy for atopic eczema by specialist nurses. Brit J Dermatol 1999; 141 (Suppl 55): 102-7.
(6) Cork M J C, Murphy R, Carr J, Battle D, Ward S, Båvik C and Tazi-Ahnini R. The rising prevalence of atopic eczema and environmental trauma to the skin. Dermatology in Practice 2002; 10 (3): 22-26
(7) Cork M. J., Timmins J., Holden C., Carr J. Berry V., Ward S. J., Tazi-Ahnini R. (2003) An audit of adverse drug reactions to aqueous cream in children with atopic eczema. Pharmaceutical Journal 271: 746-747
(8) Elias P M. Epidermal lipids, barrier function and desquamation. J. Invest. Dermatol. 1983; 80: (6) 44-49.
(9) Flohr C, Johansson S G, Wahlgren C F, Williams H. How atopic is atopic dermatitis? J Allergy Clin Immunol. 2004 July; 114(1): 150-8.
(10) Hachem J P et al. The Journal of Investigative Dermatology: The Society for Investigative Dermatology—Abstracts, 2005. 66th Annual Meeting, May 4-7 2005, St Louis, Mo. April 2005/Volume 124/Number 4 (Supplement). Pg A58.
(11) Kao J S, Fluhr J W, Man M Q et al. Short-term glucocorticoid treatment compromises both permeability barrier homeostasis and stratum corneum integrity: inhibition of epidermal lipid synthesis accounts for functional abnormalities. J Invest Dermatol 2003; 120: 456-464.
(12) Murphy R, Williams H C, Duff G W, Cork M J, Total and specific IgE and definitions of atopy. Br. J. Dermatol. 141 (suppl.):25, 1999.
(13) Thestrup-Pedersen K. The incidence and pathophysiology of atopic dermatitis. J Eur Acad Dermatol Venereol 1996; 7 (suppl 1): 53-57.
(14) White M I, McEwan Jenkinson D, Lloyd D H. The effect of washing on the thickness of the stratum corneum in normal and atopic individuals. Br. J. Dermatol. 1987 116: 525-30.
(15) Williams H C. Is the prevalence of atopic dermatitis increasing? Clinical and Experimental Dermatology 1992; 17: 385-391
(16) Spergel J M and Paller A S. Atopic dermatitis and the atopic march. J. Allergy Clin. Immunol. Vol 112, No. 6 (2003)
(17) Cork M. J. et al New perspectives on epidermal barrier dysfunction in atopic dermatitis: Gene-environment interactions. J Allergy Clin Immunol. 2006 July; 118(1) 3-21.
(18) Cork M. J. et al Predisposition to sensitive skin and atopic eczema. Community Practitioner 2005 December; 78(12) 440-442.

1. A composition for topical administration to the skin for treating and/or preventing a dermatological condition involving an abnormal decrease in the cell-to-cell adhesion between epithelial cells comprising a zinc salt which is at least partially soluble in water and an acid, wherein the composition has a pH less than or equal to about 6.

2-42. (canceled)

43. A composition according to claim 1, wherein the acid is an alpha-hydroxy carboxylic acid or a carboxylic acid.

44. A composition according to claim 1, wherein the acid is selected from the group consisting of citric acid, glyceric acid, glycolic acid, picolinic acid, tartaric acid, pantothenic acid, lactic acid, gluconic acid, pyruvic acid, salicylic acid, formic acid, acetic acid, propionic acid and butyric acid.

45. A composition according to claim 1, wherein the zinc salt has a solubility in water of at least 0.01 moles/l, at least 0.05 moles/l, at least 0.1 moles/l, or between 0.01 moles/1 and 0.6 moles/l, or between 0.05 moles/1 and 0.4 moles/l.

46. A composition according to claim 1, wherein the zinc salt is selected from the group consisting of zinc citrate, zinc glycerate, zinc glycolate, zinc picolinate, zinc tartrate, zinc pantothenate, zinc lactate, zinc gluconate, zinc pyruvate, zinc salicylate, zinc formate, zinc acetate, zinc propionate and zinc butyrate.

47. A composition according to claim 1, wherein the acid is lactic acid, and wherein the zinc salt is zinc lactate.

48. A composition according to claim 1, wherein the pH is equal to or less than about 5.5, or in the range of 4.0 to 5.5.

49. A composition according to claim 1, which is in the form of a soak, ointment, cream, lotion, paste, gel, stick, spray, aerosol, bath oil, shampoo, soap, foam, spray or solution.

50. A composition according to claim 1, further comprising one or more pharmaceutically acceptable excipient, carrier, diluent or vehicle.

51. A composition according to claim 50, wherein the excipients include those that provide emolliency, skin hydration via humectancy and occlusion and aesthetic qualities.

52. A composition according to claim 50, wherein the excipient is selected from the group consisting of petrolatum, light liquid paraffin, mineral oils, fatty acid derivatives including isopropyl myristate, isopropyl palmitate and such, glycerides, natural oils and lanolins, such as Medilan.

53. A composition according to claim 50, wherein an oil phase is petrolatum-mineral oil as this gives maximum occlusion percent of composition.

54. A composition according to claim 50, wherein an excipient is selected from the group consisting of volatile and non-volatile silicone and alkyl esters.

55. A composition according to claim 1, wherein the dermatological condition is selected from the group consisting of eczema, dermatitis, atopic eczema, atopic dermatitis, non-atopic eczema, non-atopic dermatitis, seborrheic eczema, irritant contact dermatitis, allergic contact dermatitis, pruritus and sensitive skin, or wherein the dermatological condition is selected from the group consisting of irritant/allergic contact eczema or dermatitis of the hands and irritant/allergic facial eczema or dermatitis.

56. A composition for topical administration to the skin for treating and/or preventing a dermatological condition involving an abnormal decrease in the cell-to-cell adhesion between epithelial cells comprising zinc lactate and lactic acid, and wherein the composition has a pH of about 4.0 to 5.0.

57. A method of treating a dermatological condition involving an abnormal decrease in the cell-to-cell adhesion between epithelial cells comprising topical administration of a composition according to claim 1.

58. A method according to claim 57, wherein the dermatological condition is selected from the group consisting of eczema, dermatitis, atopic eczema, atopic dermatitis, non-atopic eczema, non-atopic dermatitis, seborrheic eczema, irritant contact dermatitis, allergic contact dermatitis, pruritus and sensitive skin, or wherein the dermatological condition is selected from the group consisting of irritant/allergic contact eczema or dermatitis of the hands and irritant/allergic facial eczema or dermatitis.

59. A composition as defined in claim 1, adapted for the slowing, prevention or prophylaxis of the atopic march, and/or reducing TH1 to TH2 switching.

60. A composition as claimed in claim 59, adapted for controlling the development, preventing or reducing the risk of development, or prophylaxis of asthma and/or rhinitis.

61. A method for the slowing, prevention or prophylaxis of the atopic march, and/or reducing TH1 to TH2 switching, comprising use of the composition as defined in claim 1.

62. A method according to claim 61, for controlling the development, preventing or reducing the risk of development, or prophylaxis of asthma and/or rhinitis.

63. A composition comprising zinc lactate and lactic acid, wherein the composition has a pH of about 4.0 to 5.0, optionally wherein zinc lactate is present at about 2% or at about 3%.

64. A method of generating a composition according to claim 1, comprising contacting zinc oxide and an acid.

65. A method according to claim 64, wherein the acid is selected from the group consisting of citric acid, glyceric acid, glycolic acid, picolinic acid, tartaric acid, pantothenic acid, lactic acid, gluconic acid, pyruvic acid, salicylic acid, formic acid, acetic acid, propionic acid and butyric acid.

66. A set of materials for use in generating a composition according to claim 1, comprising zinc oxide and an acid.

67. A set of materials according to claim 66, wherein the acid is selected from the group consisting of citric acid, glyceric acid, glycolic acid, picolinic acid, tartaric acid, pantothenic acid, lactic acid, gluconic acid, pyruvic acid, salicylic acid, formic acid, acetic acid, propionic acid and butyric acid.
